(19) United States
(12) Patent Application Publication
(10) Pub. No.: US 2024/0058264 A1
(43) Pub. Date: Feb. 22, 2024

(54) PHARMACEUTICAL FORMULATIONS OF NINTEDANIB FOR INTRAOCULAR USE

(71) Applicant: Boehringer Ingelheim International GmbH, Ingelheim am Rhein (DE)

(72) Inventors: Paul ASHTON, San Diego, CA (US); Jianbing CHEN, Wayland, MA (US); Florian Andreas COLBATZKY, Biberach an der Riss (DE); Hong GUO, Wayland, MA (US); Laibin LUO, Ridgefield, CT (US); Kerstin Julia SCHAEFER, Danbury, CT (US)

(21) Appl. No.: 18/234,400

(22) Filed: Aug. 16, 2023

(30) Foreign Application Priority Data
Aug. 16, 2022 (EP) 22190482.4

(51) Int. Cl. A61K 9/00; A61K 31/496; A61K 47/32

(52) U.S. Cl. CPC A61K 9/0051; A61K 31/496; A61K 47/32

(57) ABSTRACT

Formulations and intravitreal implants containing nintedanib can be used in methods for the treatment of back-of-the-eye diseases.

### FIELD OF THE INVENTION

This invention relates to pharmaceutical formulations of nintedanib suitable for intraocular use and treatment of retinal conditions, more specifically to long-term sustained-release pharmaceutical formulations of nintedanib, to intravitreal (IVT) implants formed from such formulations, to methods for preparation of such formulations and IVT implants as well as to the use of such formulations and IVT implants in methods for the treatment of certain back-of-the-eye diseases.

### BACKGROUND OF THE INVENTION

In the treatment of severe chronic ocular diseases, especially in the case of degenerative retinal conditions such as wet age-related macular degeneration (wAMD), dry macular degeneration, geographic atrophy, diabetic macular edema (DME) or nonproliferative diabetic retinopathy (NPDR), cystoid macular edema (CME), choroidal neovascularization (CNV), and retinal vein occlusion, implantable sustained-release delivery devices or implantable sustained-release formulations that would continuously administer a therapeutic agent to the eye for a prolonged period of time are desirable alternatives to the burdensome regimen of intravitreal injections of therapeutic agents which have to be repeated regularly after relative short intervals, e.g., monthly.

Age-related macular degeneration (AMD) is a common disease and the leading cause of severe visual loss in the population over 50 years of age in the western world. AMD compromises central vision, as it leads to degeneration and irreversible damage of the macula. There are mainly two common forms of AMD: dry AMD (also called non-exudative AMD) and wet AMD (also called exudative or neovascular form, characterized by an abnormal growth of new blood vessels). About 90 percent of the AMD cases relate to the dry form, affecting the central retina-macula area that enables us to see (in particular fine details), to read, and to differentiate colors. In contrast, only about ten percent of people with AMD have the wet form; however, wAMD can quickly cause severe visual loss as new abnormal choroidal and retinal blood vessels leak and bleed and destroy the architecture of the retina, which leads to photoreceptors cell death. Those rapidly growing abnormal blood vessels, known as choroidal neovascular membranes (CNVM), and the changes in blood vessels permeability and exudation have been treated with repeatable intravitreal injections of anti-VEGF agents.

Angiogenesis is implicated in the pathogenesis of intraocular neovascular diseases such as proliferative retinopathies and AMD; therefore, the therapeutic use of inhibitors of vascular endothelial growth factor receptors (VEGFRs) for treatment of these diseases is a known approach in the art, as described in WO 2006/047325 and elsewhere.

Nintedanib, (3-Z-[1-(4-(N-((4-methyl-piperazin-1-yl)-methylcarbonyl)-N-methyl-amino)-anilino)-1-phenyl-methylene]-6-methoxycarbonyl-2-indolinone), the compound of formula A,

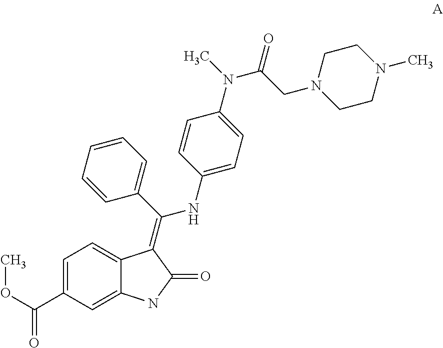 [SMILES: COC(=O)c1ccc2c(c1)NC(=O)/C2=C(\Nc1ccc(N(C)C(=O)CN2CCN(C)CC2)cc1)c1ccccc1]

is a highly potent, orally bioavailable intracellular tyrosine kinase inhibitor. It inhibits vascular endothelial growth factor receptors (VEGFRs), platelet-derived growth factor receptors (PDGFRs) and fibroblast growth factor receptors (FGFRs). It binds competitively to the adenosine triphosphate (ATP) binding pocket of these receptors and blocks intracellular signaling. In addition, nintedanib inhibits Fms-like tyrosine-protein kinase 3 (Flt 3), lymphocyte-specific tyrosine-protein kinase (Lck), tyrosine-protein kinase lyn (Lyn) and proto-oncogene tyrosine-protein kinase src (Src) (Hilberg et al., Cancer Res. 2008, 68, 4774-4782). Hence, its kinase specificity profile comprises kinases related to angiogenesis, fibrosis, inflammation, and proliferation. Thus, nintedanib has valuable pharmacological properties, e.g., for the treatment of immunologic diseases or pathological conditions involving an immunologic component, for oncological or fibrotic diseases.

Nintedanib is described in WO 01/27081. WO 2004/013099 discloses its monoethanesulphonate (esylate) salt which is especially suitable for development as a medicament; further salt forms are presented in WO 2007/141283. Pharmaceutical dosage forms comprising nintedanib are disclosed e.g., in WO 2009/147212 and WO 2009/147220. The use of nintedanib for the treatment of immunologic diseases or pathological conditions involving an immunologic component is described in WO 2004/017948, the use for the treatment of oncological diseases is described in WO 2004/096224 and the use for the treatment of fibrotic diseases is described in WO 2006/067165.

Sustained-release formulations allow for the delivery of a drug over a prolonged period of time. Their mode of administration and their release kinetics can have a profound effect on therapeutic efficacy. The use of polymer materials in this regard is well established and has led to numerous successful methods of both controlling drug release and providing sustained release over days to months.

Polymeric drug delivery devices for implantation in vivo have demonstrated durability and biocompatibility. Many of these drug delivery devices which provide sustained release of an agent, however, are inert under biological conditions, which results in the need for surgical resection following complete drug release, particularly in case of larger devices. An additional challenge is the difficulty in manufacturing materials with therapeutic agents and particularly maintaining uniformity among miniaturized devices with dimensions in mm scale or smaller. There remains a need for biodegradable or bioerodible implantable drug delivery devices with controlled drug release that can be manufactured to consistent specifications on mm or sub-mm scale.

Intravitreal implants have been developed which deliver a sustained concentration of drug over a period of time. These implants are injected or surgically implanted in the vitreous of the eye for the sustained release of drug to the posterior part of the eye. Many matrix-based sustained release drug delivery systems are known to be suitable for intraocular, in particular intravitreal placement for prolonged treatment of back-of-the-eye indications. The field of sustained drug delivery is well described and there are many technologies. For sustained release from implants in which a drug is released by diffusion through a matrix, the release kinetics are typically defined by Fick's laws of diffusion. Higuchi later described solutions to Fick's laws pertaining to diffusion from ointments (Higuchi T, Physical chemical analysis of percutaneous absorption process from creams and ointments, J Soc Cosmet. Chem 1960; 1:85-97). These are directly applicable to drug release from solid systems such as drugs in polymer matrices. Higuchi also described the situation where a drug is not dissolved in the matrix material but exists as particles within the matrix (Higuchi T, Release of medicaments from ointment bases containing drugs in suspension, J Pharm Asci. 1961: 50:874-875).

In both cases diffusion is a function of the square root of time, i.e., the amount of drug released over a period of time is a function of the square root of the time period. In some pharmaceutical dosage forms this is adequate, but for others it is desirable that the drug is released at a more constant rate, e.g., approximately following zero-order kinetics over a large part of the release time.

In an attempt to achieve more linear release, implants can be prepared from matrices of drug in bioerodible polymers. “Bioerodible polymer” means a polymer which does not degrade in vivo. Bioerodible polymer matrices provide the advantage over biodegradable polymer matrices that they erode mechanically by solubilization and are excreted unchanged whereas biodegradable polymers are cleaved in the body to monomers which may cause toxicology issues. Many biodegradable implants have been prepared, frequently containing polylactic acid and polyglycolic acid or copolymers (PLGA). The rationale is that from these systems drug will be released as the implant breaks down rather than by diffusion through the polymer matrix. In practice this has been difficult to achieve. For any shaped implant, the surface area becomes smaller as the implant erodes so even in a perfect system, release would be non-linear. In addition, diffusion of drug from the device still occurs, particularly for water soluble drugs or for implants with a high drug content (over 10%). One example of this is the Ozurdex® implant (Allergan). This implant comprises dexamethasone in a PLGA matrix and is purported to be a 6 months device. In practice it releases over 99% of its drug in the first month (Chang-Lin J-E, Attar M, Acheapong A A et al. Pharmacokinetics and pharmacodynamics of a sustained-release dexamethasone intravitreal implant, Invest Ophth Vis Sci 2011; 52:80-86). Another issue with these systems is that PLGA undergoes self-catalyzed breakdown, the net effect of which is a slow initial degradation followed by a rapid “bulk” erosion resulting in so called S kinetics, i.e. an initial release rate determined by diffusion through the matrix which slows down (square root time kinetics) followed by a faster release as the device disintegrates. One attempt to provide more linear release was to prepare implants with low drug loading (less than 5%) in the shape of wafers, so the surface area would not change significantly as they erode. Devices of this type were commercialized as Gliadel®. In vitro they gave very linear long term release but in-vivo data indicated that release occurs via both diffusion and erosion and the wafers are fully depleted in 5 days (Flemming A B, Saltzman W M, Pharmacokinetics of the carmustine implant, Clin Pharmacokinetics 41 (6) 403-419 (2002)).

Non-erodible implants such as Vitrasert®, Retisert® and ILUVIEN® provide more constant release rates. In these systems a central drug core like a tablet or a paste (in a polymer matrix) is encased in an impermeable polymer. Drug release occurs across a small hole (diffusion port) in the impermeable layer and can be further controlled by coatings of permeable polymers over the diffusion port. When immersed in water (or placed in the eye) water diffuses into the central drug core and dissolves some of the drug which then diffuses through the diffusion port. The amount of water entering the core is small as the amount of drug dissolved in this internal water is also small, thus the release rate is low. Further, as long as excess drug is present in the drug core the solution of drug in the core will be saturated and the concentration gradient across the diffusion port will be constant, giving linear release. While these systems have the advantage of providing relatively linear release, they are neither bioerodible nor biodegradable.

There remains great need in the medical field for alternative treatments of back-of-the-eye indications such as a sustained-release delivery system releasing a therapeutic concentration of the active on long-term scale from an intravitreal implant directly to the posterior of the eye with a high safety and efficacy profile. Moreover, any sustained-release implant is highly dependent on the selection of polymers, co-polymers, drug-polymer interaction, load uniformity, porosity, size, surface-area to volume ratio, and the like for providing its drug release and degradation characteristics and the manufacturing techniques used in the prior art implants can induce inherent drawbacks in each of these parameters.

U.S. Pat. No. 5,378,475 describes a sustained-release implant for insertion into the vitreous of the eye. The implant has a first impermeable coating, such as ethylene vinyl acetate, surrounding most, but not all, of a drug reservoir and a second permeable coating, such as a permeable crosslinked polyvinyl alcohol, disposed over the first coating including the region where the first coating does not cover the drug reservoir, to provide a location through which the drug can diffuse out of the implant. The implant also has a tab, which can be used to suture the device in place in the eye. The implant devices are prepared by applying coating solutions, such as by dipping, spraying, or brushing, of the various coating layers around the drug reservoir.

U.S. Pat. No. 8,871,241 discloses an injectable sustained release drug delivery device having a cylindrical cross-section including a core containing one or more drugs and one or more polymers. The core may be surrounded by a polymer outer layer. The device is formed by extruding or otherwise preforming a polymeric skin for a drug core and the drug core may be co-extruded with the skin or inserted into the skin after the skin has been extruded, and possibly cured. Polymers to form the skin and the core include poly(caprolactone), ethylene vinyl acetate polymer, poly(ethylene glycol) (PEG), polyvinyl alcohol (PVA), poly(lactic acid) (PLA), poly(glycolic acid) (PGA), poly(lactic-co-glycolic acid) (PLGA), polyalkyl cyanoacrylate, polyurethane, nylons, or copolymers thereof.

The device may have an outside diameter suitable for injection in the vicinity of a patient's eye, as either an intraocular or periocular injection, with a 30-gauge (0.3 mm outer diameter according to EN ISO 9626) needle to about a 12-gauge (2.7 mm outer diameter according to EN ISO 9626) needle, or with a needle ranging in inside diameter from about 0.0055 inches (0.1397 mm) to about 0.0850 inches (2.159 mm).

The device may be formed by combining at least one polymer, at least one drug, and at least one liquid solvent to form a liquid suspension or solution wherein, upon injection, such suspension or solution undergoes a phase change and forms a gel. The configuration may provide for controlled release of the drug(s) for an extended period.

The device may be fabricated as an extended mass that is segmented into drug delivery devices, which may be left uncoated so that the drug core is exposed on all sides or (where a skin is used) at the ends of each segment or coated with a layer such as a layer that is permeable to the drug, semi-permeable to the drug, impermeable, or bioerodible.

A vast range of drugs is disclosed which may be incorporated into the devices, including among many others, angiogenesis suppressors, antiproliferative compounds and tyrosine kinase inhibitors.

WO 02/074196 discloses ocular implants which administer a therapeutic drug to the eye according to dual mode release kinetics by initial delivery of a “loading dose” at a high release rate soon after placement of the implant in or near the eye, as a first administration mode, followed by drug delivery via a continuous, sustained lower release rate thereafter, as a second maintenance dosage administration mode, and within the same treatment regimen using the same implant device. The implants include:
(a) a composite material matrix layer including:
   (i) a therapeutic agent, and
   (ii) a polymeric matrix material into which the therapeutic agent is dispersed, including
      (1) a polymer permeable to the therapeutic agent and present as a bioerodible solid matrix structure, and
      (2) a water-soluble polymer having greater water solubility than the permeable polymer, and
(b) optionally, a discrete solid core containing additional therapeutic agent, which is surrounded and covered by the composite material matrix layer.

The permeable polymer may be uncrosslinked superhydrolyzed PVA, which permits diffusion of the therapeutic agent therethrough, and forms a slowly bioerodible solid structure, releasing the drug by surface erosion of the PVA and by diffusion, and the water-soluble polymer may be a pharmaceutical grade cellulose ether. The rate of erosion of the superhydrolyzed PVA is sufficiently slow that the polymer material in the implant will dissolve so that the therapeutic agent will disintegrate only after an extended period of time and provide a slow sustained delivery of drug. The superhydrolyzed polyvinyl alcohol may be a polyvinyl alcohol having at least 98.8 wt % hydrolysis and a weight average molecular weight of about 85,000 to about 150,000. Heating the matrix implants at temperatures above 100° C. will encourage PVA crosslinking. This may be desirable when attempting to reduce drug release rates from a particular implant and also in controlling the rate at which the implant erodes.

Embodiments of this implant can be installed in the vitreous humor to deliver 2-methoxyestradiol for treatment of CNVM but many other therapeutic agents and drugs are disclosed which can be delivered by the implants, amongst others angiogenesis compounds such as VEGF antagonists.

WO 2005/110362 discloses a drug delivery system for treating ocular conditions comprising at least one bioerodible implant suitable for insertion into an ocular region, comprising (i) an active agent, and (ii) a bioerodible polymer, wherein the bioerodible implant should release a therapeutic level of the active agent into the ocular region for a time period between about 30 days and about 1 year. Preferably the active agent is an anti-inflammatory agent, the bioerodible polymer is a PLGA co-polymer. The drug delivery system may comprise a plurality of bioerodible implants, each bioerodible implant having a unique drug release profile, preferably up to three implants implantable in a posterior ocular region. The implants may be prepared using extrusion methods.

WO 2006/039271 discloses a process for making a plurality of drug delivery devices for implantation in the eye of a patient that are made in part of polyvinyl alcohol using a consistent curing process resulting in less variation in the drug release rate from one device to the next.

WO 2018/054077 discloses methods of treating an ocular disease, including front- and back-of the eye indications, comprising administering an effective amount of a pharmaceutical composition to the eye of a subject in need thereof, wherein the pharmaceutical composition is a topical formulation, such as eye drops, and comprises nintedanib, or a salt thereof. The disclosure also relates to pharmaceutical composition or formulation, which can be used for treating ocular diseases.

WO 2020/219890 discloses sustained-release biodegradable ocular hydrogel implants for treatment of back-of-the-eye indications, comprising a tyrosine kinase inhibitor (TKI), including nintedanib and others, a polymer network comprising a plurality of polyethylene glycol (PEG) units, and a clearance zone, wherein the clearance zone is devoid of undissolved TKI particles prior to release of the TKI.

WO 2020/243608 discloses an implant comprising a tyrosine kinase inhibitor and a bioerodible polyester polymer, providing sustained release of a small molecule tyrosine kinase inhibitor, such as axitinib, from the bioerodible polyester polymer implant for the treatment of ophthalmic indications, such as neovascular age related macular degeneration and diabetic macular edema, by intravitreal injection of the implant. The implant is designed to be pre-loaded into a small diameter needle and injected via self-sealing scleral needle penetration at the pars plana.

WO 2017/083779 and WO 2020/102758 disclose optionally biodegradable solid aggregating microparticles, having a mean diameter e.g., between 20 and 40 microns, for long-term therapy of an ocular disorder, comprising an effective amount of a therapeutic agent such as the VEGFR inhibitor sunitinib, for injection towards the bottom of the vitreous body. The microparticles are composed of polymers such as PLGA, PLGA-PEG or PLA and aggregate in vivo to form at least one pellet of at least 500 μm that provides sustained drug delivery in such a manner that the pellet stays substantially outside the visual axis as not to significantly impair vision.

### BRIEF DESCRIPTION OF THE DRAWINGS

FIG. 1:

Average release profile (n=6) of nintedanib esylate in PBS at 37° C. over 270 days for implant I-6.

FIG. 2:

Release profiles of nintedanib esylate in PBS at 37° C. over 45 days for implants which had been dried for 3 hours at 130° C.

FIG. 3:

Release profiles of nintedanib esylate in PBS at 37° C. over 45 days for implants which had been dried for 3 hours at 150° C.

FIG. 4:

Release profiles of nintedanib esylate in PBS at 37° C. over 127 days for implants (that disintegrated before day 80) which had been dried for 1 hour at 100° C.

FIG. 5:

Release profiles of nintedanib esylate in PBS at 37° C. over 135 days for implants which had been dried for 3 hours at 130° C.

FIG. 6:

Daily release profile nintedanib esylate in PBS at 37° C. over 135 days for implants which had been dried for 3 hours at 130° C.

FIG. 7:

Extrudate comparison between nintedanib esylate (1), nintedanib mixture of 20% esylate and 80% free base (2) and nintedanib free base (3) according to E)2) preparation of strands (I-6) above, with 12% (w/v) PVA solution before oven heating

FIG. 8:

Extrudate comparison between nintedanib esylate (1), nintedanib mixture of 20% esylate and 80% free base (2) and nintedanib free base (3) according to E)2) preparation of strands (I-6) above, with 12% (w/v) PVA solution before oven heating

### SUMMARY OF THE INVENTION

In a first aspect, the invention relates to a long-term sustained-release pharmaceutical formulation of the API comprising 80 to 95% (w/w) of API and 5 to 20% (w/w) of PVA.

In a second aspect, the invention relates to a coated or uncoated IVT implant having a body consisting of a long-term sustained-release pharmaceutical formulation of the API according to the first aspect of the invention, the body optionally having a polymer coating.

In a third aspect, the invention relates to a method for the preparation of a long-term sustained-release pharmaceutical formulation of the API according to the first aspect of the invention, the method comprising the steps of
a) preparing an aqueous PVA solution, and
b) mixing the PVA solution with API powder.

In a fourth aspect, the invention relates to a method for the preparation of an IVT implant according to the second aspect of the invention, the method comprising the steps of
a) preparing an aqueous PVA solution,
b) mixing the PVA solution with API powder,
c) loading the mixture into an extrusion device,
d) extruding the mixture through an extruder head to form an extrudate strand,
e) optionally allowing the extrudate strand to dry,
f) heating the extrudate strand,
g) cutting the extrudate strand into implant pieces of identical lengths, and
h) sterilizing the implant pieces so obtained,
   wherein the method may further comprise an optional step of coating the extrudate strand after step d), e), or f) or the implant pieces after step g) in a solution of coating polymer.

In a fifth aspect, the invention relates to a method for the treatment of a back-of-the-eye disease in a patient in need thereof, the method being characterized in that a pharmaceutical formulation according to the first aspect of the invention is administered to the eye of the patient, in particular at least one IVT implant according to the second aspect of the invention is implanted into the vitreous of the eye of the patient.

Also, the invention relates to a pharmaceutical formulation according to the first aspect of the invention, in particular to an IVT implant according to the second aspect of the invention, for use in a method for the treatment of a back-of-the-eye disease in a patient in need thereof.

Also, the invention relates to the use of the API in the manufacture of a pharmaceutical formulation according to the first aspect of the invention, in particular of an IVT implant according to the second aspect of the invention, for the treatment of a back-of-the-eye disease in a patient in need thereof.

Other aspects of the present invention will become apparent to the person skilled in the art directly from the foregoing and following description.

### General Terms and Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to.

The terms “treatment” and “treating” as used herein embrace both therapeutic, i.e., curative and/or palliative, and preventative, i.e., prophylactic, treatment.

Therapeutic treatment refers to the treatment of patients having already developed one or more of said conditions in manifest, acute or chronic form. Therapeutic treatment may be symptomatic treatment to relieve the symptoms of the specific indication or causal treatment to reverse or partially reverse the conditions of the indication or to stop or slow down progression of the disease.

Preventative treatment (“prevention”) refers to the treatment of patients at risk of developing one or more of said conditions, prior to the clinical onset of the disease to reduce said risk.

The terms “treatment” and “treating” include the administration of one or more active compounds to prevent or delay the onset of the symptoms or complications and to prevent or delay the development of the disease, condition, or disorder and/or to eliminate or control the disease, condition or disorder as well as to alleviate the symptoms or complications associated with the disease, condition or disorder.

The term “therapeutically effective amount” means an amount of a compound of the present invention that (i) treats or prevents the particular disease or condition, (ii) attenuates, ameliorates, or eliminates one or more symptoms of the particular disease or condition, or (iii) prevents or delays the onset of one or more symptoms of the particular disease or condition described herein.

The expression “intraocular use” refers to the use within the eye. It encompasses i.a. intravitreal, suprachoroidal, intracameral, and subconjunctival use.

“Long-term sustained-release” of a drug means release of a drug over an extended period of time, e.g., over weeks or months, in particular over more than 3, 6, 9, or 12 months.

The expression “API” and “nintedanib” mentioned herein regarding any aspect of the invention or in the context of the invention is meant to be interchangeable and to include the group consisting of nintedanib (free base), a pharmaceutically acceptable nintedanib salt and a blend consisting of nintedanib (free base) and a pharmaceutically acceptable nintedanib salt. Amounts provided herein are generally expressed in relation to nintedanib esylate, if not indicated otherwise.

The term “inactive ingredients” means any component other than an active ingredient.

The phrase “pharmaceutically acceptable” is employed herein to refer to those compounds, excipients, materials, compositions, and/or dosage forms which are, within the scope of sound medical judgment, suitable for use in contact with the tissues of human beings and animals without excessive toxicity, irritation, allergic response, or other problem or complication, and commensurate with a reasonable benefit/risk ratio.

Pharmaceutically acceptable excipients suitable for the preparation of pharmaceutical formulations, e.g., for IVT implants, will be known to those skilled in the art.

The terms “bioerodable” and “biodegradable” refer to the gradual disintegration, dissolution, or breakdown of a polymer, formulation, or implant over a period of time in a biological system, e.g., by one or more physical or chemical degradative processes like dissolution by solubilization, enzymatic processes, or hydrolysis.

Biodegradable polymers are typically liable to hydrolysis under physiological conditions due to the presence of hydrolytically and/or enzymatically cleavable functional groups (e.g., anhydride, ester, amide bonds). Biodegradation can result in polymer backbone scission or cleavage of water-soluble side chains. The cleavage products can then be metabolized and excreted, resulting in complete removal.

Bioerodible polymers erode mechanically via biological processes that solubilize the polymer and enable absorption into the surrounding tissue.

A paste, as used herein, describes a thick wet mixture of a solid, e.g., an API, and a solution, e.g., a PVA solution.

Viscosity measurements are made according to JPE Monographie of Polyvinylalcohol (Fully Hydrolyzed Polyvinyl Alkohol; Viscosity), with a 4% solution in water, at 20° C., using a rotational viscometer.

### DETAILED DESCRIPTION OF THE INVENTION

There is a medical need to improve options for treatment of degenerative or persistent back-of-the-eye conditions by providing implantable sustained-release delivery devices continuously releasing a therapeutic agent to the eye for a prolonged period of time, for example, time periods of therapeutic phases of at least 3 months, at least 6 months, at least 9 months, at least 12 months or more, with a release rate suitable to maintain a therapeutic drug level at a desired posterior ocular region or site.

Numerous matrix-based drug delivery systems have been developed to date, however the implants according to the invention provide the following unique combination of properties:
1) the release of the drug is dependent on the surface area of the device which declines only slightly during the therapeutic phase as drug is released;
2) the primary function of the polymer matrix (or coating if one is used) is not to provide a diffusional barrier to the drug's release but substantially to maintain the surface area of the implant;
3) the release rate declines so slowly throughout the duration of the release, so that therapeutic concentrations of the drug are maintained in the vitreous body of the eye throughout the extended treatment phase;
4) the matrix is bio-erodible (not biodegradable);
5) the erosion rate can be adjusted by a heat curing process so that the implant structure breaks down after over 90% of the drug has been released;
6) these implants are able to maintain release and drug concentration in the vitreous body within the therapeutic window of the drug while using relatively low concentrations of polymer in the matrix (drug:polymer ratio (w/w) of >1:1, more typically >4:1 and 6:1 (w/w), e.g., up to 10:1 or 20:1).

In addition, as has been shown by light stress and long-term stability studies, the implants according to the invention are suitable for long-term storage in that they provide adequate release, physical and chemical stability properties over months.

The formulation principle underlying the invention is to develop a delivery system with minimum addition of pharmaceutical excipients and few steps in the manufacturing process. The pharmaceutical excipient selected is polyvinyl alcohol (PVA). This material has a well-known safety profile and has been used in several approved intra-ocular products. The low percentage of PVA (not more than 20% (w/w)) in the formulation allows a high drug loading in the product. PVA dissolves in water into a viscous solution at desired concentration which can be mixed with API to form a paste.

The formulation process involves the steps of mixing of API with PVA solution and extruding the paste, e.g., through a needle tip. The extruded strand is then heat-treated, e.g., in an oven. Depending on the temperature and time, this process changes PVA crystallinity in the matrix which subsequently changes the drug's dissolution rate in the wet environment.

The nintedanib IVT implant according to the invention is bioerodible and provides long-term almost constant sustained release of nintedanib for intraocular use. It has a very high drug:polymer ratio (greater than 80% drug loading by weight). In ophthalmology, these features are particularly attractive as implants should be small enough to be injected into the eye via an incision that is sufficiently small that it does not need to be closed after the injection. This requires injecting via a needle with a diameter equal to or smaller than that of a 22-gauge needle. Thus, a high drug content is extremely important.

Bioerodible IVT implants are particularly advantageous for treatment of diseases such as macular degeneration and diabetic macular edema which are not considered curable but must be treated for the duration of the patient's life. Non-erodible implants would accumulate in the eye unless removed (a nontrivial procedure). Erodible implants do not cause this issue. Biodegradable polymers have the potential issue that the monomers released by degradation of the polymers have the potential to be inflammatory (Evaluation of the toxicity of intravitreally injected PLGA microspheres and rods in monkeys and rabbits: effects of depot size on inflammatory response. Thackberry E A, Farman C, Zhong F et al., Invest Ophthalmol Vis Sci (2017) 58:4274-4285). Bioerodible polymers slowly dissolve and the polymer itself does not break down.

Many eye diseases must be treated continuously, therefore maintenance of therapeutic concentrations of nintedanib as provided by the IVT implants according to the invention is a substantial advantage, particularly for drugs with a potentially small therapeutic window.

In a first aspect, the invention relates to a long-term sustained-release pharmaceutical formulation of the API comprising 80 to 95% (w/w) of API and 5 to 20% (w/w) of PVA.

According to one embodiment, said pharmaceutical formulation consists of 80 to 95% (w/w) of API, 5 to 20% (w/w) of PVA (w/w), optionally pharmaceutically acceptable inactive ingredients and optionally traces of water.

Traces of water may be present in said pharmaceutical formulation, e.g., because of incomplete drying of the formulation. They normally relate to amounts of not more than 1% (w/w).

Preferably, said pharmaceutical formulation consists of 80% to 95% (w/w) of API and 5 to 20% of PVA (w/w).

According to another embodiment, said pharmaceutical formulation comprises 85% to 95% of API and 5% to 15% of PVA (w/w), preferably 87% to 91% of API and 9% to 13% of PVA (w/w).

Preferably, said pharmaceutical formulation consists of 85% to 95% of API and 5% to 15% of PVA (w/w), preferably 87% to 91% of API and 9% to 13% of PVA (w/w), and optionally traces of water. For instance, the mass ratios of API:PVA may be 100:5, 100:7, 100:8, 100:10, 100:15, 100:16.5, or 100:18, in particular 100:10 and 100:15, most preferably 100:15. In particular, mass ratios of 100:15, 100:16.5, and 100:18 provide pastes that can still be extruded easily (see the fourth aspect of the invention) and form pharmaceutical formulations and implants with increased long-term integrity, i.e. with later disintegration, and long-term drug release in phosphate buffered saline (PBS).

For the preparation of pharmaceutical formulations and IVT implants that provide prolonged release of a drug substance at a therapeutic level over several months, drug substances of low water solubility would be preferred to reduce the risk of premature depletion of the implant due to rapid dissolution of the drug substance. Thus, for formulations and implants according to the present invention, the free base of nintedanib with its very poor solubility in water would be a reasonable choice among different nintedanib species available. However, it has surprisingly been found that also IVT implants according to the invention that contain nintedanib esylate with its much higher water solubility of 2.8 mg/mL reveal long-term release properties that are suitable and desirable for the intended clinical use. Thus, the API in the pharmaceutical formulation according to the invention is preferably nintedanib (free base); more preferred it is a (pharmaceutically acceptable) nintedanib salt or a blend of a (pharmaceutically acceptable) nintedanib salt with nintedanib (free base), most preferably said salt is nintedanib esylate.

Pharmaceutically acceptable nintedanib salts suitable with regard to any aspect of the invention include the mono-ethanesulfonate (“esylate”) salt and nintedanib salts disclosed in WO 2007/141283, such as nintedanib chloride, bromide, phosphate, sulfate, methanesulfonate, ethanedisulfonate, benzenesulfonate, tosylate, camphorsulfonate, naphthalene-1,5-disulfonate, citrate, D-tartrate, L-tartrate, L-lactate, glycolate, glycinate, L-malate, D-malate, malonate, oxalate, benzoate, mandelate, saccharinate, salicylate and ascorbate, or the bis {3-Z-[1-(4-(N-((4-methyl-piperazin-1-yl)-methylcarbonyl)-N-methyl-aminoyanilino)-1-phenyl-methylene]-6-methoxycarbonyl-2-indolinone}-fumarate, bis {3-Z-[1-(4-(N-((4-methyl-piperazin-1-yl)-methylcarbonyl)-N-methyl-amino)-anilino)-I-phenyl-methylene]-6-methoxycarbonyl-2-indolinone}-maleate, and bis {3-Z-[1-(4-(N-((4-methyl-piperazin-1-yl)-methylcarbonyl)-N-methyl-amino)-anilino)-1-phenyl-methylene]-6-methoxycarbonyl-2-indolinone}-succinate.

Preferred nintedanib salts regarding any aspect of the invention are nintedanib esylate, chloride, bromide, phosphate, sulfate, methanesulfonate, ethanedisulfonate, benzenesulfonate, tosylate, citrate, D-tartrate, L-tartrate, L-lactate, glycolate, glycinate, L-malate, D-malate, malonate, oxalate, benzoate, mandelate, salicylate and ascorbate. Particularly preferred nintedanib salts regarding any aspect of the invention are nintedanib esylate, chloride, bromide, phosphate, sulfate, methanesulfonate, and ethanedisulfonate. Most preferred is the nintedanib esylate salt.

According to one embodiment, regarding the particle size distribution, nintedanib esylate as used for the preparation of the above-mentioned formulations should be characterized by a particle size distribution (e.g., as determined by the method described in Example A)) of D50≤20 μm and D90≤50 μm. Alternatively, nintedanib esylate may be characterized by D10≤5 μm, D50≤25 μm, D90≤50 μm, and/or D98≤60 μm; preferably, by D10≤3 μm, D50≤20 μm, D90≤40 μm, and/or D98≤50 μm.

More specifically, nintedanib esylate as used for the preparation of the above-mentioned formulations may be characterized by 1.2 μm≤D10≤2.1 μm, 9.5 μm≤D50≤14.7 μm, 24.3 μm≤D90≤31.7 μm, and 34.3 μm≤D98≤42.1 μm, e.g., by 1.4 μm≤D10≤1.7 μm, 10.5 μm≤D50≤12.7 μm, 25.4 μm≤D90≤28.7 μm, and 35.2 μm≤D98≤39.2 μm.

The PVA suitable for said pharmaceutical formulations should conform to pharmacopeial requirements (e.g., Ph. Eur., JPE). Various grades of PVA are available that differ in their degree of polymerization and their degree of hydrolysis which determine the physical properties of the different grades. They are characterized by their viscosities and ester values (characterizing the degree of hydrolysis).

It has been found that the use of PVA with a degree of hydrolysis of 88% for preparing pharmaceutical formulations according to the invention results in implants that disintegrate quickly in PBS. Implants with improved long-term release properties are obtained advantageously with PVA with a higher degree of hydrolysis, in particular about 99%.

According to one embodiment, the mean relative molecular mass of PVA lies between 20 000 and 150 000, the viscosity is 3 mPa·s to 70 mPa·s, and the ester value is not greater than 280.

Preferably, the PVA grade is characterized by
a viscosity of 23.8-32.2 mPa·s (4% PVA in aqueous solution) and
a high degree of hydrolysis 97%) or an ester value of 9-11.

More preferably, the PVA grade is characterized by
a viscosity of approximately 28 mPa·s (4% PVA in aqueous solution) and
a degree of hydrolysis of approximately 99% or an ester value of approximately 11.

According to another embodiment, only one grade of PVA is employed in said pharmaceutical formulation.

In a second aspect, the invention relates to a coated or uncoated IVT implant having a body consisting of a long-term sustained-release pharmaceutical formulation of the API according to the first aspect of the invention, the body optionally having a polymer coating.

Said long-term sustained-release pharmaceutical formulation of the API may be according to any of the embodiments described hereinbefore for the first aspect of the invention.

It has been found that implants according to the invention, e.g., containing nintedanib esylate, maintain physical integrity in wet conditions (like in PBS) for a long time even without coating. In contrast, implants with coatings may bear the risk of quick disintegration due to high osmotic pressure inside the coating membrane.

According to a preferred embodiment, the IVT implant is uncoated.

According to another embodiment, the IVT implant is coated.

Polymer coatings of IVT implants according to the invention should be biocompatible, not causing any inflammation, bioerodible or biodegradable and may be selected from the group consisting of PVA, poly(D,L-lactide-co-glycolide) (PLGA), poly-caprolactone (PCL), polylactic acid, polyglycolic acid, polyethylene adipate, and polyesteramide. According to another embodiment, the IVT implant is coated wherein the polymer coating consists of PVA. The PVA employed in the coating may be of the same grade as the PVA employed in the pharmaceutical formulation.

The diameter of IVT implants, whether coated or not, should be sufficiently small as to allow them to be inserted into the vitreous of the eye via an acceptably small incision. For most purposes this is 22 gauge or smaller, or preferably 24 gauge or smaller since larger incisions would require a suture to close the wound. Similarly, the overall length of the implants should be less than 10 mm and preferably 6 mm or less so as to reduce the likelihood that the implant will lie in the visual axis.

According to one embodiment, the IVT implant is shaped and sized such that it can be administered via an inserter having a needle no larger than 22 gauge, in particular no larger than 23, 24, 25, 26, or 27 gauge,
e.g., via an inserter having a 22-, 23-, 24-, 25-, 26-, or 27-gauge needle,
preferably via an inserter having a needle no larger than 24 gauge, e.g., a 24-gauge extra-thin wall needle.

According to another embodiment, the IVT implant is cylindrical in shape.

According to another embodiment, the diameter of the IVT implant is in the range from 0.2 to 0.4 mm, e.g., 0.25 to 0.27 mm or 0.32 to 0.35 mm.

According to one embodiment, the IVT implant is not more than 10 mm in length; for safe clinical use, it should be not more than 7 mm in length, preferably not more than 6 mm in length;
preferably it is 1 to 7 mm in length, more preferably 1.0 mm to 6.0 mm in length,
e.g., 1.0 mm, 2.5 mm, 3.5 mm, 4.0 mm, 4.5 mm, 5.0 mm, 5.5 mm, or 6.0 mm in length,
most preferably 2.5 mm, 3.5 mm, 5.0 mm, or 6.0 mm in length.

According to one embodiment, the IVT implant is characterized by a nintedanib esylate content in the range from 100 μg to 250 μg, preferably 120 μg to 160 μg or 180 μg to 220 μg, e.g., about 140 μg or about 200 μg.

According to another embodiment, the IVT implant is characterized by a nintedanib esylate content in the range from 400 μg to 600 μg, preferably 420 μg to 500 μg or 520 μg to 570 μg, e.g., about 440 μg, about 475 μg, or about 545 μg.

It has surprisingly been found that IVT implants containing nintedanib esylate as the API reveal long-term release properties that are suitable and desirable for the intended clinical use, despite the good water solubility of nintedanib esylate.

According to one embodiment, the IVT implant is characterized by an in vitro daily release of nintedanib esylate from day 10 to day 30 in the range from 1 μg/day to 2 μg/day, preferably 1.1 μg/day to 1.8 μg/day, more preferably 1.2 μg/day to 1.6 μg/day, most preferably about 1.3 μg/day (equivalent to about 1.1 μg/day of nintedanib base).

According to another embodiment, the IVT implant is characterized by an in vitro daily release of nintedanib esylate of not less than about 1.1 μg/day over 6 months, preferably not less than about 1.2 μg/day over 6 months, more preferably not less than about 1.3 μg/day over 6 months.

According to another embodiment, the IVT implant is characterized by in vitro drug release over more than 9 months.

In a third aspect, the invention relates to a method for the preparation of a long-term sustained-release pharmaceutical formulation of the API according to the first aspect of the invention, the method comprising the steps of
a) preparing an aqueous PVA solution, and
b) mixing the PVA solution with API powder.

Said long-term sustained-release pharmaceutical formulation of the API may be according to any of the embodiments described hereinbefore for the first aspect of the invention.

In step a), the aqueous PVA solution may be prepared by dissolving the desired amount of PVA in deionized water. Optionally, the mixture may be heated and/or stirred to facilitate the dissolution process.

According to one embodiment, the aqueous PVA solution prepared in step a) has a mass concentration of PVA between 2% and 20% (w/v), preferably between 8% and 15%, e.g., about 10% or about 13.6%.

In step b), the PVA solution can be mixed with the API powder by placing a weighed amount of nintedanib powder (esylate salt, base or a blend of the two), optionally in micronized form, into a mortar, then slowly adding a defined volume of the PVA solution as the paste is mixed with a pestle. On a larger scale this can be done with a commercial mixer (e.g., a Hobart mixer). A typical ratio of API to PVA solution is about 1:1 (w/v, e.g., g/mL). Step b) should be carried out until a homogeneous paste of the components is obtained.

The PVA and/or the API powder used for the preparation of said pharmaceutical formulation preferably exhibit the characteristics described hereinbefore for the first aspect of the invention.

Thus, the API powder, e.g., nintedanib esylate, may be micronized prior to its employment in step b) of said method to meet the desired particle size distribution.

In a fourth aspect, the invention relates to a method for the preparation of an IVT implant according to the second aspect of the invention, the method comprising the steps of
a) preparing an aqueous PVA solution,
b) mixing the PVA solution with API powder,
c) loading the mixture into an extrusion device,
d) extruding the mixture through an extruder head to form an extrudate strand,
e) optionally allowing the extrudate strand to dry,
f) heating the extrudate strand,
g) cutting the extrudate strand into implant pieces of identical lengths, and
h) sterilizing the implant pieces so obtained,
   wherein the method may further comprise an optional step of coating the extrudate strand after step d), e), or f) or the implant pieces after step g) in a solution of coating polymer.

Said IVT implant may be according to any of the embodiments described hereinbefore for the second aspect of the invention.

Steps a) and b) of said method may be according to any of the embodiments described hereinbefore for the third aspect of the invention. Also, the API powder, e.g., nintedanib esylate, may be micronized prior to its employment in step b) of said method. Preferably, after mixing, the obtained mixture should not be held in an open container for more than 15 minutes in order not to negatively impact the extrudability of the paste.

The extrusion device of step c) comprises an extrusion head (or extrusion tip) as mentioned in step d).

The extrusion device may be a syringe fitted with a needle, a single-screw extruder, a twin-screw extruder, a piston pump, or a peristaltic pump. The extruder head may be anything with an aperture sized so that the resulting extrudate strand is of the desired thickness, e.g., a needle tip.

The extrusion head may have a circular profile such that a cylindrical extrudate strand is obtained. Instead of cylindrical strands, different strand geometries may be produced, e.g., toblerone shaped, by using alternatively shaped extruder heads.

The extrusion tip may have different sizes, and the tip's internal diameter defines the implant's outer diameter. It has been found that extrusion using 23-gauge or 22-gauge tips produces implants (of 0.25-0.27 mm and 0.32-0.35 mm, respectively, in diameter) that fit well into a 24-gauge extra-thin wall needle (inner diameter of about 0.37 mm) which is particularly suitable for the intended clinical use.

Thus, the extrusion head should be of 21 gauge or smaller, e.g., 22 or 23 gauge, preferably 22 gauge, so that the final implant can be injected into the eye via an inserter no larger than 22 gauge, e.g., 23 or 24 gauge, preferably 24 gauge, in particular via a 24-gauge extra-thin wall needle. Thus, after processing, the implant pieces can be injected directly into the eye via an incision that is sufficiently small that is does not need to be closed after the injection.

According to one embodiment, the extrusion head has a circular profile.

According to one embodiment, the inner diameter of the extrusion head is not more than about 0.5 mm, preferably not more than about 0.4 mm, e.g., about 0.33 mm or about 0.41 mm.

According to one embodiment, the extrudate strand is obtained as a continuous strand of cylindrical shape.

According to another embodiment, the extrudate strand is of 0.2 to 0.4 mm in diameter, e.g., 0.23-0.29 mm or 0.30-0.37 mm.

For step d), it has surprisingly been found that mixtures prepared from nintedanib esylate or from blends of nintedanib esylate with nintedanib free base (e.g., in a ratio of at least 1:9 (w/w), e.g., 1:9 or 2:8) according to steps a)-b) can be extruded much more easily than mixtures prepared from nintedanib free base only. An example of this is shown in FIG. 8.

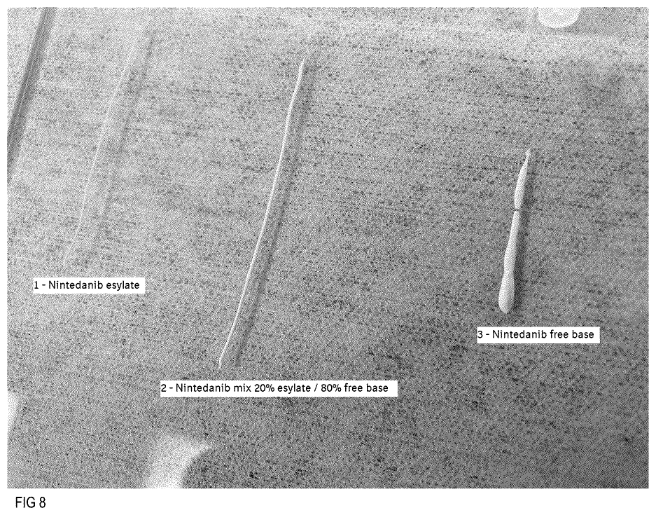

In addition, extrudate strands formed from mixtures containing nintedanib esylate reveal good physical properties: For instance, such mixtures form continuous strands of advantageous consistency and cohesiveness, i.e. they do not break apart easily during the extrusion process. Also, after extrusion and already prior to any drying or heating step, the extrudate strands show good dimensional stability, in particular they do not flatten under the influence of gravity but maintain their circular profile and cylindrical shape very well. An example of this is shown in FIG. 7.

In optional step e), the extrudate strand is allowed to air dry for an appropriate time.

When step e) is performed at ambient temperature, 35° C., or 50° C., no significant variation has been observed in the measured implant diameter and the in vitro releases of the implants were comparable.

According to a preferred embodiment, step e) is performed without additional heating, e.g., at ambient temperature.

According to another embodiment, step e) is performed at elevated temperatures, e.g., at 35° C., or at 50° C.

According to another embodiment, the extrudate strand is allowed to dry for about 30 min to about 24 hours, preferably for at least 2 hours, e.g., from about 2 hours to about 14 hours, e.g., for 2, 4, 6, 10 or 12 hours.

In a preferred embodiment, the extrudate strand is allowed to dry for at least 2 hours at ambient temperature.

Step f) represents a heat curing step. This step aims at increasing the crystallinity and hardness of the PVA to reduce the dissolution rate of the API from the implant and to improve implant integrity.

According to one embodiment, the heating is performed for at least 2 hours, preferably for at least 3 hours, e.g., for about 3 hours, about 4 hours, about 5 hours, or about 8 hours.

According to another embodiment, the extrudate strand is heated to temperatures between 100° C. and 180° C., preferably between 120° C. and 160° C., e.g., to about 130° C., about 140° C., about 150° C., or about 160° C., more preferably between 130° C. and 150° C.

In a preferred embodiment, the heating is performed for at least 2 hours at a temperature between 120° C. and 180° C., more preferably for at least 3 hours at about 130° C. to 150° C., most preferably for about 3 hours at about 130° C.

In step g), the extrudate strand is cut into implants of identical lengths by appropriate means.

Suitable lengths of the implants are described hereinbefore for the second aspect of the invention.

According to one embodiment, the extrudate strand is cut into implant pieces of identical lengths of not more than 10 mm.

According to a preferred embodiment, a 23 G extrudate strand is cut into implant pieces of identical lengths of 1 to 7 mm, e.g., 2.5, 4.0, 5.0, or 6.0 mm.

According to another preferred embodiment, a 22 G extrudate strand is cut into implant pieces of identical lengths of 1 to 7 mm, e.g., 3.5, 4.5, 5.0 or 6.0 mm, preferably of 5 or 6 mm.

In the sequence of method steps described herein, step g) does not necessarily have to be carried out after step f). As an alternative to the sequence described hereinbefore, step g) can be carried out also before step f) or even before optional step e).

In step h), sterilization of the implant pieces may be achieved by gamma or e-beam irradiation.

According to one embodiment, sterilization is achieved by gamma irradiation.

According to one embodiment, gamma irradiation may be performed with doses of up to 25 kGy, e.g., about 15 kGy, about 20 kGy, or about 25 kGy, e.g., at 22.5-27.5 kGy.

No significant differences in the release rate of implants before and after gamma irradiation have been observed over 10 weeks.

Instead of or in addition to step h), said method for the preparation of an IVT implant may be carried out under aseptical conditions.

According to a preferred embodiment, the fourth aspect of the invention relates to a method for the preparation of an IVT implant according to the second aspect of the invention, the method comprising the steps of
a) preparing an aqueous PVA solution with a mass concentration of PVA between 8% and 15% (w/v), by dissolving PVA in deionized water, optionally with heating and/or stirring,
b) mixing the PVA solution with nintedanib esylate powder,
c) loading the mixture into an extrusion device,
d) extruding the mixture through an extruder head of 22 or 23 gauge to form an extrudate strand, preferably as a continuous strand of cylindrical shape and a diameter of 0.2 to 0.4 mm,
e) optionally allowing the extrudate strand to dry at ambient temperature,
f) heating the extrudate strand for at least 2 hours at a temperature between 120° C. and 180° C.,
g) cutting the extrudate strand into implant pieces of identical lengths of 1 to 7 mm, and
h) sterilizing the implant pieces so obtained,
   wherein the extrudate strand obtained after step d), e), or f) or the implant pieces obtained after step g) optionally are coated in a solution of coating polymer.

The sequence of steps g) and f) is interchangeable, step g) can be carried out before or after step f) or even before optional step e).

In a fifth aspect, the invention relates to a method for the treatment of a back-of-the-eye disease in a patient in need thereof, the method being characterized in that a pharmaceutical formulation according to the first aspect of the invention is administered to the eye of the patient.

Likewise, the invention relates to a method for the treatment of a back-of-the-eye disease in a patient in need thereof, the method being characterized in that at least one IVT implant according to the second aspect of the invention is implanted into the vitreous of the eye of the patient.

Also, the invention relates to a pharmaceutical formulation according to the first aspect of the invention for use in a method for the treatment of a back-of-the-eye disease in a patient in need thereof.

Likewise, the invention relates to an IVT implant according to the second aspect of the invention for use in a method for the treatment of a back-of-the-eye disease in a patient in need thereof.

Also, the invention relates to the use of the API in the manufacture of a pharmaceutical formulation according to the first aspect of the invention for the treatment of a back-of-the-eye disease in a patient in need thereof.

Likewise, the invention relates to the use of the API in the manufacture of an IVT implant according to the second aspect of the invention for the treatment of a back-of-the-eye disease in a patient in need thereof.

Said back-of-the-eye disease may be selected from the group consisting of wet age-related macular degeneration (wAMD), dry macular degeneration, geographic atrophy, diabetic macular edema (DME), nonproliferative diabetic retinopathy (NPDR), cystoid macular edema (CME), choroidal neovascularization (CNV), retinal vein occlusion, and retinitis pigmentosa,
preferably from the group consisting of wAMD, DME or retinal vein occlusion; most preferably said back-of-the-eye disease is wAMD.

Said method for the treatment may comprise one-time or repeated implantation of an IVT implant according to the second aspect of the invention in the vitreous of the patient's eye.

According to one embodiment, the method of treatment comprises repeated implantation of an IVT implant according to the invention in the vitreous of the patient's eye.

According to another embodiment, said implantation of an IVT implant is carried out using a 24-gauge extra-thin wall needle.

The time interval between repeated implantations should be at least 3 months or at least 6 months, preferably at least 9 months or at least 12 months, e.g., 9, 10, 11, 12, 13, 14, or 15 months, most preferably the time interval between repeated implantations is 12 months.

At each implantation, one or more IVT implants may be implanted in the vitreous of the patient's eye.

According to a preferred embodiment, the method for the treatment of a back-of-the-eye disease is characterized in that only one IVT implant is implanted into the vitreous of the eye of the patient at a time.

### EXAMPLES AND EXPERIMENTAL DATA

The following examples are for the purpose of illustration of the invention only and are not intended in any way to limit the scope of the present invention.

### A) Method for Measuring Particle Size Distribution

The particle size distribution is determined via laser diffraction. A laser diffraction sensor (e.g., helium-neon laser optical system HELOS from Sympatec) with dry dispersion unit (e.g., RODOS from Sympatec) and a vibratory feeding unit (e.g., VIBRI from Sympatec) may be employed with the following settings:
semi-circular multi-element photo-detector with 31 channels
focal length: 100 mm (measuring range 0.5/0.9-175 μm)
time basis: 100 ms
start: 0 s after opt. conc. ≥2.0%, always applicable; stop: 3 s after opt. conc. ≤1.0% or after 30 s real time
pressure: 2.0 bar; vacuum: max.
mode: HRLD (High Resolution Laser Diffraction Mode)
opt. concentration: 2-20%
feed rate: 80%; feed height: 1.3 mm

Results are reported as average of 3 independent sample measurements.

### B) Pharmaceutical Formulations

| | relative amounts (mass ratios) of ingredients in formulation | | | | | | |
|---|---|---|---|---|---|---|---|
| | Example | Example | Example | Example | Example | Example | Example |
| | F-1 | F-2 | F-3 | F-4 | F-5 | F-6 | F-7 |
| Nintedanib | 100 | 100 | 100 | 100 | 100 | 100 | 100 |
| Esylate | | | | | | | |
| PVA | 5 | 7 | 8 | 10 | 15 | 16.5 | 18 |
| Deionized | — | | — | — | — | — | — |
| Water* | | | | | | | |

*removed during the preparation process, only traces (i.e. not more than about 1% (w/w) remaining in the formulation)

### C) Bioerodible Implants

| | characteristics of nintedanib bioerodible implants | | | | | |
|---|---|---|---|---|---|---|
| | Example | Example | Example | Example | Example | Example |
| | I-1 | I-2 | I-3 | I-4 | I-5 | I-6 |
| Formulation | F-4 | F-4 | F-3 | F-4 | F-5 | F-5 |
| inner diameter of tip | 0.330 | 0.330 | 0.406 | 0.406 | 0.406 | 0.406 |
| from which extruded [mm] | (23 G tip) | (23 G tip) | (22 G tip) | (22 G tip) | (22 G tip) | (22 G tip) |
| Length [mm] | 2.5 | 3.5 | 3.5 | 5.0 | 5.0 | 6.0 |
| Target drug content | 140 | 200 | 320 | 475 | 440 | 545 |
| [μg nintedanib esylate] | | | | | | |

### D) General Process for the Preparation of Coated and Uncoated Bioerodible Nintedanib Implants

Bioerodible nintedanib implants according to the invention are prepared in a three-step process:

1) Preparation of PVA Solution

PVA is weighed and placed in a glass conical flask. Deionized water is then added, and the mixture heated on a hot plate with stirring by magnetic stirrer, the top of the flask is covered with a glass plate or foil to reduce evaporation. When the PVA has fully dissolved the heat is turned off and the solution allowed to cool to room temperature with constant stirring. When cool the volume is adjusted with deionized water to make up for any loss from evaporation. Typical mass concentrations (w/v) of PVA in the final solution are between 2% and 20%, usually about 10% or about 13.6%. It is also preferable that the PVA be Ph. Eur./JPE grade with a viscosity of 23.8-32.2 mPa·s (4 PVA in aqueous solution) and a high degree of hydrolysis (≥97%).

2) Preparation of Implants

A weighed amount of micronized nintedanib powder (esylate salt, base or a blend of the two), typically nintedanib esylate powder, is placed into a mortar, a solution of PVA is then slowly added as the paste is mixed with a pestle. On a larger scale this can be done with a commercial mixer (e.g., Hobart). Typical ratios of nintedanib to PVA solution are about 1:1, e.g., 100 g of nintedanib powder are mixed with 100 mL of PVA solution.

The paste is then loaded into a syringe fitted with a needle. The paste is pushed through the needle tip onto a glass plate to form a continuous line (strand). The internal diameter of the needle dictates the diameter of the strand. The strand is allowed to air dry for over 2 hours before being heated in an oven at between 100° C. and 180° C. for up to 6 hours, preferably at 130° C. to 135° C. for 2 to 4 hours, e.g., for 3 hours. After removal from the oven the strand is cut to length.

Alternatively, the strand can be coated in a solution of PVA, allowed to dry again and then heated. Other options include heating the dried paste line before dip coating and then reheating or dip coating after implants have been cut to length.

3) Loading into Applicators, Packaging and Sterilization

Implants are loaded into the barrel of a customized applicator. The external diameter of the barrel should be no larger than, and ideally smaller than a 22-gauge needle. After the end of the barrel is sealed to prevent the implant falling out, the applicator is packaged to prevent the accidental depression of the plunger or if spring loaded, the accidental activation of the spring. The assembly is then pouched for sterilization by gamma (e.g., up to 25 kGy) or e-beam. Pouching can be either single of, ideally double pouched (to allow the inner, sterile pouch to be placed in a sterile field immediately prior to use).

### E) Specific Process for the Preparation of Uncoated Bioerodible Nintedanib Implants According to C)

A specific preparation process is described in the following exemplarily for I-6, but may be applied analogously for I-3, I-4, I-5, and in a similar fashion for further implants according to the invention, e.g., for I-1 and I-2.

1) Preparation of PVA Solution

10% (w/v) and 13.6% (w/v) PVA solutions are prepared by adding 10 g and 13.6 g, respectively, of PVA (Merck Germany, 99% hydrolysed) to 100 mL of water for injection in a glass flask. The flask is placed on a heated stir plate and heated until the PVA is fully dissolved. After cooling, water is then added with stirring to bring up to weight (to correct for evaporation of water during the heating).

2) Preparation of Implants (I-6)

In a mixing bowl 5 g of nintedanib esylate is added to 5.5 mL of the 13.6% (w/v) PVA solution and mixed with a stainless-steel spatula to obtain a smooth, uniform paste. The paste is then transferred into 1 mL syringes fitted with a 22-gauge blunt tip. The loaded syringe is then placed in a manual Arbor press such that the distal end of the 22-gauge tip is facing down. The arm of the press is lowered to make contact with the syringe plunger and then, with constant force not to exceed 40 N pushed down to extrude the API/PVA paste through the 22-gauge tip to form an approximately 20 cm strand. The syringe is then removed from the press and the strand laid flat onto a smooth surface and removed from the tip. This can then be repeated depending on the required batch size.

The extrudate strand is allowed to air dry for at least 2 hours before being placed in an oven heated to 130° C. for 3 hours. After cooling, the strands are cut to 6.0 mm in length using a cutting fixture and razor blade. Implants are visually inspected for deformities and extraneous particular matter.

3) Loading into Applicators, Packaging and Sterilization

Implants are then loaded into the applicator which consists of a 24-gauge XTW (extra-thin wall) needle on an inserter body (similar to a syringe but with a pusher rod rather than a plunger where the pushed rod is fitted into the distal end of the needle). A single (or optionally two) implant are loaded into the distal end of the needle and the applicator cap fitted over the end so as to 1) shield the sharp end of the needle and 2) prevent the implant from falling out of the needle. A removable clip is placed on the pusher rod to prevent its accidental depression and the assembly then placed in a foil pouch and sealed with a bar heat sealer. The loaded foil pouch is then placed in a Tyvek (spunbonded HDPE fiber material) pouch which is heat sealed with a bar heat sealer. The double pouched sealed system is then gamma (25 kGy) sterilized.

### F) Specific Process for the Preparation of Coated Bioerodible Nintedanib Implants

Coated implants similar to the uncoated ones of E) can be prepared by taking the air-dried API/PVA strand of step 2) and dipping it into an aqueous solution of 2% (w/v) or 5%(w/v) PVA. After allowing to air dry for 12 hours the coated strands are then heated in an oven at 130° C. for 3 hours and then processed in the same manner as in E). This results in cylindrical implants the walls of which are coated in a layer of PVA while the ends are uncoated.

G) Method for Measuring Drug Release Rate from Implants In Vitro

Implants are individually placed into vials containing 20 mL of PBS in a water bath at 37° C. Samples are taken periodically, e.g., once daily, and assayed by HPLC using a C-18 reverse phase column with UV detection and buffer is replaced to ensure sink conditions are maintained. Release rates per day are calculated from the HPLC results and are given as μg of nintedanib esylate per day.

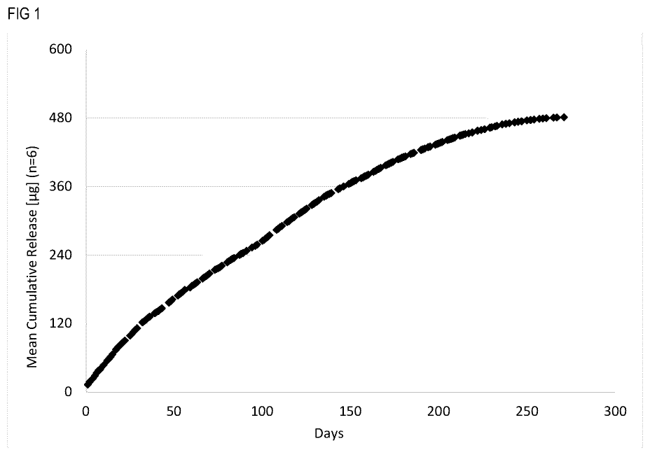

An exemplary release profile is shown in FIG. 1 for the implant I-6 as described hereinbefore.

### H) Influence of Formulation Properties, Implant Properties and Process Parameters on the Drug Release Rate

The effects of various parameters on the release rate have been determined.

1) Effect of Implant Dimensions

Uncoated nintedanib esylate implant prototypes of formulation F-4 (nintedanib esylate:PVA 100:10 (w/w)) were prepared according to the processes described herein. 22- and 23-gauge needles, respectively, were used for extrusion and strands were cut to lengths of 1.0 mm or 3.5 mm. Release rates were determined according to the process described herein. For each implant, the cumulative amount of drug released was measured over a number of days and the average release rate per mm² surface area was calculated.

The results show that, for a given API:PVA ratio, the nintedanib release rate of the implants according to the invention is proportional to their surface area.

| | Formulation Example F-4 | | |
|---|---|---|---|
| | (nintedanib esylate:PVA 100:10 (w/w)) | | |
| Size of tip from which extruded | 23 | 23 | 22 |
| [gauge] | | | |
| Length [mm] | 1.0 | 3.5 | 3.5 |
| Release Rate (average from | 0.60 | 1.83 | 2.43 |
| day 1 to 45) [μg/day] | | | |
| Surface Area [mm²] | 1.25 | 3.92 | 4.77 |
| Release Rate per surface area | 0.48 | 0.47 | 0.51 |
| [μg/(day*mm²)] | | | |

2) Effect of PVA Content

Uncoated nintedanib esylate implant prototypes of formulations F-4 and F-7, respectively, (nintedanib esylate:PVA mass 100:10 (w/w) and 100:18 (w/w), respectively) were prepared according to the processes described herein. They were extruded through 23-gauge needles, heated for 3 hours at 130° C., and cut to a length of 3.5 mm. Release rates were determined according to the process described herein.

As can be seen, there is some reduction in release rate with higher PVA content.

| | Formulation | Formulation |
|---|---|---|
| | Example F-4 | Example F-7 |
| | (nintedanib | (nintedanib |
| | esylate:PVA | esylate:PVA |
| | 100:10 (w/w)) | 100:18 (w/w)) |
| Release Rate (average | 1.82 ± 0.08 (n = 5) | 1.51 ± 0.14 (n = 6) |
| from day 1 to 50) | | |
| [μg/day] | | |

3) Effect of Temperature During Oven Drying

Uncoated nintedanib esylate implant prototypes of formulations F-4 and F-7, respectively, (nintedanib esylate:PVA mass 100:10 (w/w) and 100:18 (w/w), respectively) were prepared according to the processes described herein. They were extruded through 23-gauge needles, heated as shown below, and cut to a length of 3.5 mm. Release rates were determined according to the process described herein.

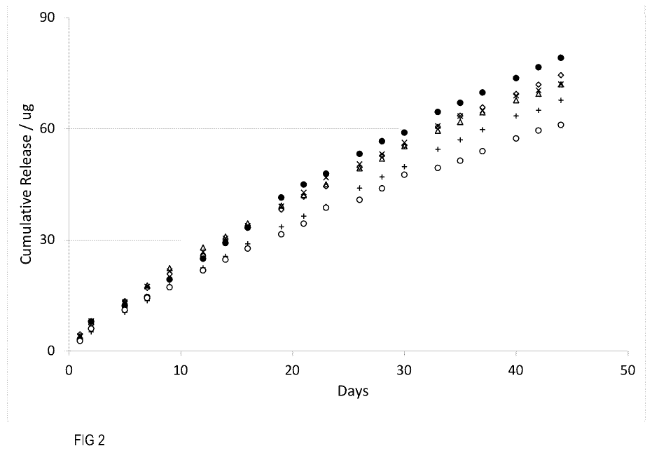

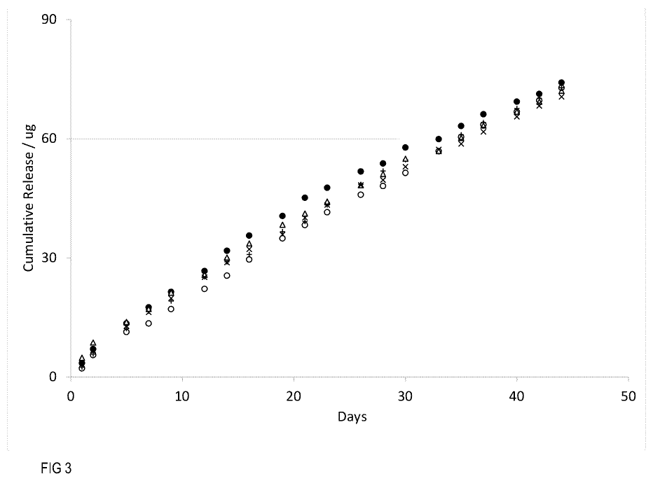

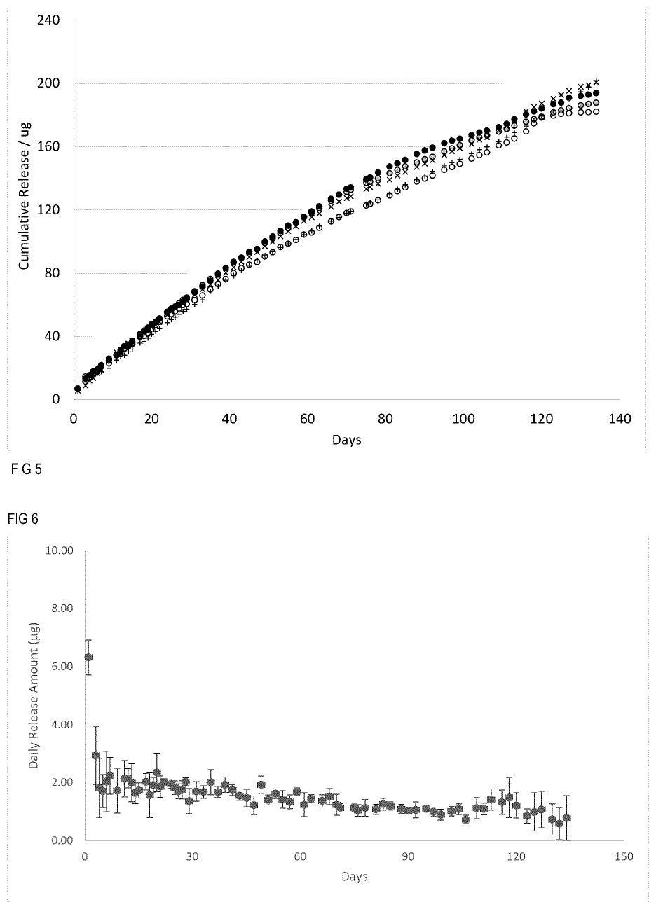

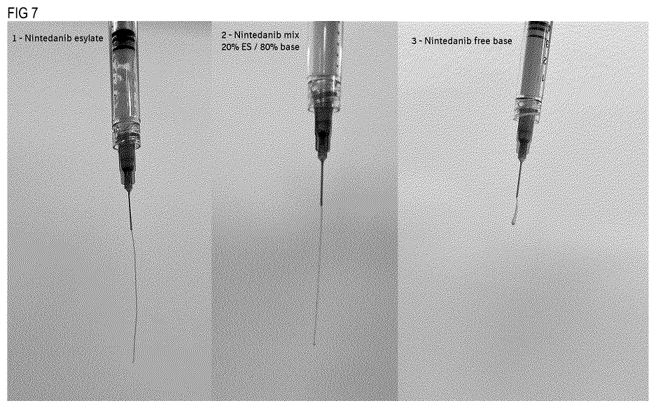

As can be seen from FIG. 2 and FIG. 3 as well as from the following table (each showing data for implants of formulations F-7), release is relatively constant, and the curing temperature has no significant effect on release rates. FIG. 6 additionally shows a surprisingly very low initial burst release, that is, below 5% after in the first two days, which contributes favourable towards a relatively constant release.

| | Formulation Example F-7 | |
|---|---|---|
| | (nintedanib esylate:PVA 100:18 (w/w) | |
| Size of tip from which extruded | 23 | 23 |
| [gauge] | | |
| Length [mm] | 3.5 | 3.5 |
| Curing Temperature over 3 | 130° C. | 150° C. |
| hours | | |
| Release Rate (μg/day) | 1.51 ± 0.14 (n = 6) | 1.54 ± 0.5 (n = 5) |
| (average from day 1 to 45) | | |

Similar graphs have been obtained for implants (23 G extrusion needle, 3.5 mm length) of formulation F-4 (nintedanib esylate:PVA 100:10 (w/w)).

Formulation Example F-4
(nintedanib esylate:PVA 100:10 (w/w))

| | Release Rate (μg/day) |
|---|---|
| Curing Temperature and Duration | (average from day 1 to 120) |
| 100° C., 1 h | 1.50 ± 0.09 |
| 110° C., 3 h | 1.37 ± 0.05 |
| 120° C., 3 h | 1.54 ± 0.20 |
| 130° C., 3 h | 1.45 ± 0.06 |

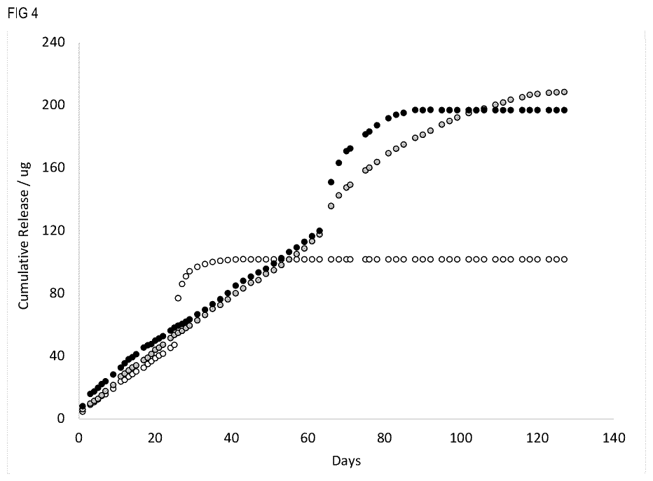

While the curing temperature did not affect the release rate, it did affect the disintegration rates of the implants. Implants heated at lower temperatures and shorter periods of time frequently broke up during dissolution testing (the table above provides the release rate for those devices that did not disintegrate). When devices disintegrate there is a marked increase of drug release followed by a plateau of the measured release rate (see e.g., FIG. 4 and FIG. 5).

The disintegration of implants heated for 1 hour at 100° C. appears to be somewhat random, but all (n=6) implants disintegrate by 120 days (FIG. 4 showing only the 3 implants disintegrating before day 80; the overall seemingly low release of one implant is an artefact due to the early disintegration of this implant.). Implants that are heated for 3 hours to 130° C. are much less likely to disintegrate over the time course of drug release. (Note: The total drug content of these implants is approximately 200 μg. Thus, near linear release is maintained until the implants are largely depleted.)

1. A pharmaceutical formulation comprising 80 to 95% (w/w) of nintedanib, or a pharmaceutically acceptable salt thereof, and 5 to 20% (w/w) of poly polyvinyl alcohol (PVA).

2. The pharmaceutical formulation according to claim 1, wherein the formulation consists of 80 to 95% (w/w) of nintedanib, or a pharmaceutically acceptable salt thereof, 5 to 20% (w/w) of PVA (w/w) and optionally traces of water.

3. The pharmaceutical formulation according to claim 1, wherein nintedanib is in the form of a pharmaceutically acceptable nintedanib salt or a blend of a pharmaceutically nintedanib salt with nintedanib free base.

4. The pharmaceutical formulation according to claim 1, wherein the PVA is characterized by a high degree of hydrolysis (≥97%) and optionally by a viscosity of 23.8-32.2 mPa·s as determined for a 4% PVA aqueous solution.

5. A coated or uncoated intravitreal (IVT) implant having a body consisting of a long-term sustained-release pharmaceutical formulation according claim 1, the body optionally having a polymer coating.

6. The IVT implant according to claim 5, wherein the implant is cylindrical in shape, 0.2-0.4 mm in diameter and not more than 7 mm in length.

7. The IVT implant according to claim 5, wherein the implant is characterized by a nintedanib esylate content in the range from 400-600 μg and/or by an in vitro daily release of nintedanib esylate of not less than about 1.1 μg/day over 6 months.

8. A method for the preparation of the pharmaceutical formulation according to claim 1, the method comprising the steps of
a) preparing an aqueous PVA solution, and
b) mixing the PVA solution with a powder of nintedanib, or a pharmaceutically acceptable salt thereof.

9. The method according to claim 8, wherein in step a) the PVA solution has a mass concentration of PVA between 8% and 15% (w/v) and/or in step b) the ratio of PVA solution to nintedanib, or a pharmaceutically acceptable salt thereof powder is about 1:1 (w/v).

10. The method according to claim 8, further comprising the steps of
c) loading the mixture into an extrusion device,
d) extruding the mixture through an extruder head to form an extrudate strand,
e) optionally allowing the extrudate strand to dry,
f) heating the extrudate strand,
g) cutting the extrudate strand into implant pieces of identical lengths, and
h) sterilizing the implant pieces so obtained,
wherein the method may further comprise an optional step of coating the extrudate strand after step d), e), or f) or the implant pieces after step g) in a solution of coating polymer.

11. The method according to claim 10, wherein the extrusion head has a circular profile and its inner diameter is not more than about 0.4 mm.

12. The method according to claim 11, wherein in optional step e) the extrudate strand is allowed to dry at ambient temperature for at least two hours and/or in step f) the extrudate strand is heated for at least 2 hours to temperatures between 120° C. and 180° C.

13. A method for the treatment of a back-of-the-eye disease in a patient in need thereof, the method comprising administering the pharmaceutical formulation according to claim 1 to the eye of the patient.

14. The method according to claim 13, wherein the back-of-the-eye disease is selected from the group consisting of wet age-related macular degeneration (wAMD), dry macular degeneration, geographic atrophy, diabetic macular edema (DME), nonproliferative diabetic retinopathy (NPDR), cystoid macular edema (CME), choroidal neovascularization (CNV), retinal vein occlusion, and retinitis pigmentosa.

15. The method according to claim 3, wherein said pharmaceutically acceptable nintedanib salt is nintedanib esylate having a particle size distribution of D50≤20 μm and D90≤50 μm.

16. A method for the treatment of a back-of-the-eye disease in a patient in need thereof, the method comprising implanting at least one IVT implant according to claim 5 into the vitreous of the eye of the patient.

17. The method according to claim 16, wherein the method comprises repeated implantation of one IVT implant using a 24-gauge extra-thin wall needle, and the time interval between repeated implantations is at least 9 months.
